(19) United States
(12) Patent Application Publication
(10) Pub. No.: US 2006/0135793 A1
(43) Pub. Date: Jun. 22, 2006

(54) Process for the dimerisation of levulinic acid, dimers obtainable by such process and esters of such dimers

(76) Inventors: Robert Wiegbertus Blessing, Amsterdam (NL); Leonardus Petrus, Amsterdam (NL)

Correspondence Address: SHELL OIL COMPANY, P O BOX 2463, HOUSTON, TX, 772522463, US

(21) Appl. No.: 11/283,985

(22) Filed: Nov. 21, 2005

(30) Foreign Application Priority Data
Nov. 26, 2004 (EP) 04106107.8

(51) Int. Cl. C07D 307/34

(52) U.S. Cl. 549/318; 554/124

(57) ABSTRACT

The present invention provides a process for dimerisation of levulinic acid, wherein an organic phase comprising levulinic acid is contacted, in the presence of hydrogen, with a strong acidic heterogeneous catalyst comprising a hydrogenating metal, at a temperature in the range of from 60 to 170° C. and a pressure in the range of from 1 to 200 bar (absolute). The invention further relates to dimers of levulinic acid and their esters, to a mixture comprising levulinic acid and its dimers obtainable by such process and to an ester mixture obtainable by esterifying such mixture of levulinic acid and its dimers.

### CROSS-REFERENCE TO RELATED APPLICATIONS

This application claims priority from European Patent Application No. 04106107.8, filed on Nov. 26, 2004, which is incorporated herein by reference.

### FIELD OF THE INVENTION

The present invention provides a process for dimerisation of levulinic acid, to dimers of levulinic acid and their esters, to a mixture comprising levulinic acid and its dimers obtainable by such process and to an ester mixture obtainable by esterifying such mixture of levulinic acid and its dimers.

### BACKGROUND OF THE INVENTION

There is an increasing demand for chemical compounds, chemical intermediates and fuels that are derived from biomass. The use of fuel components that are derived from biomass is stimulated by regulatory and fiscal promotion of biofuels, such as for example Directive 2003/30/EC of the European Parliament and Council. Esters and other derivatives from levulinic acid are promising candidates for bio-derived fuel components. Such esters have been proposed as biofuels or bio-derived fuel components. Reference is for example made to WO 94/21753, WO 03/002696, and WO 03/085071. The boiling points of the esters of levulinic acid that are typically mentioned as fuel component, such as methyl and ethyl levulinate, is such that they are more suitable as gasoline component than as diesel component. For diesel, components with a higher molecular weight would be preferred.

### SUMMARY OF THE INVENTION

It has now been found that two levulinic acid molecules can be dimerised to form a novel di-carboxylic acid in a single-stage process combining aldolcondensation, dehydration and hydrogenation. The single-stage process comprises contacting an organic phase comprising levulinic acid, in the presence of hydrogen, with a strong acidic heterogeneous catalyst comprising a hydrogenating metal, at elevated temperature and preferably at elevated pressure. The catalyst and process conditions are similar to those used in the known single-step process for the conversion of acetone into methyl isobutyl ketone (MIBK), which is also known as 4-methyl-2-pentanone. The organic phase may further comprise one or more ketones other than levulinic acid or aldehydes. In that case, not only levulinic acid dimers, but also mixed dimers formed by the reaction of levulinic acid with the other ketone or aldehyde and, provided that the ketone or aldehyde bears a proton on the carbon atom adjacent to the carbonyl group (a so-called alpha proton), also homo-dimers of two molecules of the other ketone or aldehyde, are formed.

Accordingly, the present invention provides a process for dimerisation of levulinic acid, wherein an organic phase comprising levulinic acid is contacted, in the presence of hydrogen, with a strong acidic heterogeneous catalyst comprising a hydrogenating metal, at a temperature in the range of from 60 to 170° C. and a pressure in the range of from 1 to 200 bar (absolute).

In the process according to the invention, two different keto-di-carboxylic acids are formed by aldolcondensation of two levulinic acid molecules and subsequent dehydration and hydrogenation of the aldol dimer. The two resulting keto-di-carboxylic acids have the molecular structure 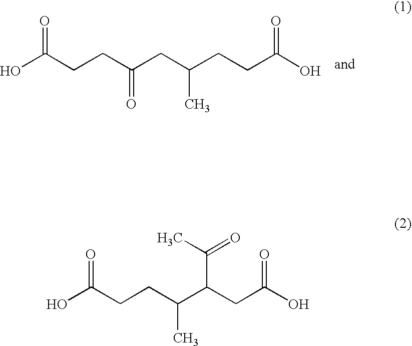
respectively.

In the process according to the invention, part of the aldol dimers formed from two molecules of levulinic acid form an internal ester bond, resulting in two different C₁₀ compounds with one carboxyl group and one lactone group. The two resulting compounds have the molecular structure 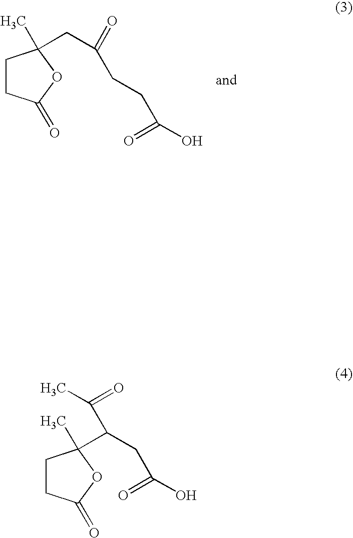
respectively.

A small part of the lactones according to formulas (3) and (4) may be further converted into a C₁₀ compound with two lactone groups. This results in two different compounds with the molecular structure 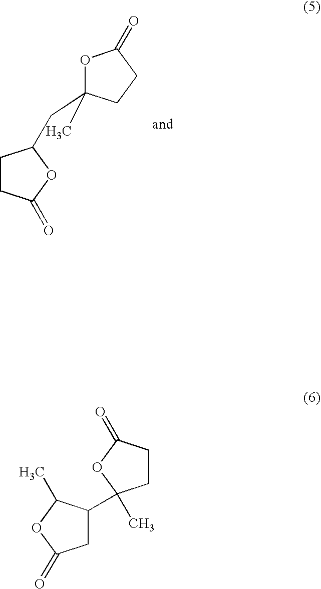
respectively.

A small part of the di-carboxylic acids according to formula (1) or (2) may be further converted into a C₁₀ compound with a carboxyl group and a lactone group by internal ester formation followed by hydrogenation. This results in two different compounds with the molecular structure 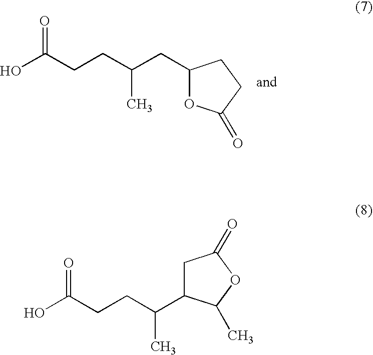
respectively.

Accordingly, the invention also provides a di-carboxylic acid having the molecular formula (1) or (2), a C₁₀ compound with a carboxyl group and a lactone group having the molecular structure according to formula (3), (4), (7) or (8) and a C₁₀ compound with two lactone groups having the molecular structure according to formula (5) or (6).

In the case that also a ketone other than levulinic acid or an aldehyde is present in the organic phase, also mixed dimers formed from one molecule of levulinic acid and one molecule of other ketone or aldehyde are formed.

Accordingly, the invention further provides a mixed dimer formed from one molecule of levulinic acid and one molecule of a ketone or aldehyde, obtainable by the process as hereinbefore defined.

The levulinic acid dimers and mixed dimers may each be separated from the effluent of the process and purified by techniques known in the art.

The di-carboxylic acids, the C₁₀ compounds with a carboxyl group and a lactone group, and the mono-carboxylic mixed dimers according to the invention can be used as an intermediate for useful compounds. The di-carboxylic acids can for example be used as co-monomer in polymers or converted in the corresponding di-ester. The C₁₀ compounds with a carboxyl group and a lactone group, and the mono-carboxylic mixed dimers polyesters may be converted into a mono-ester. Such di- and mono-esters of the dimers can suitably be used in a fuel composition or as solvent for biomass liquefaction.

Accordingly, the invention further provides a di-ester having the general molecular structure according to formula 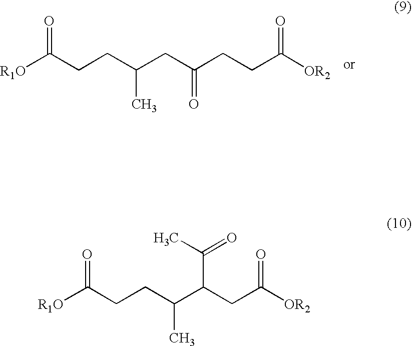
wherein R₁ and R₂ are, independently, an organic radical which is covalently connected to the oxygen atom by a carbon atom.

The invention still further provides an ester obtainable by reacting a C₁₀ compound with a carboxyl group and a lactone group as hereinbefore defined or a mixed dimer as hereinbefore defined with an alcohol under esterifying conditions, preferably an alkyl alcohol comprising up to 22 carbon atoms, more preferably an alkyl alcohol comprising 1 to 10 carbon atoms, even more preferably methanol, ethanol, 1-butanol, or 2-methyl-1-propanol.

In the process according to the invention, typically 10 to 50 wt % of the levulinic acid and, if present, equimolar amounts of the other ketone(s) or aldehyde(s) are converted into dimers. The main dimer formed is the di-carboxylic acid according to formula (1). Therefore, the process results in a mixture mainly comprising levulinic acid and the di-carboxylic acid according to molecular formula (1) and, in case that a ketone other than levulinic acid or an aldehyde is present in the organic phase, also other ketone(s) or aldehyde(s), mixed dimers, and, in case that the other ketone or aldehyde bears a proton on the carbon atom adjacent to the carbonyl group, dimers of the other ketone or aldehyde.

If the organic phase comprises no aldehyde or ketone other than levulinic acid, the whole effluent of the process according to the invention, i.e. a mixture mainly comprising levulinic acid and the di-carboxylic acid having the molecular structure of formula (1), may be reacted with an alcohol under esterifying conditions to yield a mixture comprising an ester of levulinic acid and the di-esters of the two di-carboxylic acids. Such ester mixture can suitably be used in transportation fuel, preferably in diesel fuel.

Accordingly, the invention further provides a mixture comprising levulinic acid and the di-carboxylic acid according to molecular formula (1), obtainable by the process according to the invention and to a mixture comprising an ester of levulinic acid and a di-ester of the di-carboxylic acid according to formula (1), obtainable by reacting the mixture comprising levulinic acid and the di-carboxylic acid with an alcohol under esterifying conditions, preferably an alkyl alcohol comprising 1 to 10 carbon atoms, more preferably methanol, ethanol, 1-butanol, or 2-methyl-1-propanol.

### DETAILED DESCRIPTION OF THE INVENTION

In the process according to the invention, an organic phase comprising levulinic acid is contacted, in the presence of hydrogen, with a strong acidic heterogeneous catalyst comprising a hydrogenating metal. Suitable catalysts and process conditions are similar to those used in the known single-step process for the conversion of acetone into MIBK. This single-step process for MIBK preparation is for example described in Kirk-Othmer's Encyclopedia of Chemical Technology, 3rd ed., 1981, Vol. 13, p. 909, in Ullmann's Encyclopedia of Industrial Chemistry, 5th ed., 1990, Vol. A15, p. 80 and in WO 99/65851.

The catalyst is a mixed catalyst having both an acid function and a hydrogenating function. Suitable catalysts are those known to be suitable for the acid catalysed single-step conversion of acetone into MIBK, for example cation-exchange resin/Pd. The catalyst preferably comprises a hydrogenating metal on a strong acidic cation-exchange resin, for example Amberlyst® CH-28. The catalyst may comprise a hydrogenating metal on an acidic zeolite material, such as for example protonated zeolite beta or ZSM-5.

The hydrogenating metal may be any hydrogenating metal known in the art. Preferably the hydrogenating metal is a Group VIII metal, more preferably Ni, Pd, or Pt, even more preferably palladium. The catalyst may comprise more than one hydrogenating metal.

The organic phase and hydrogen are contacted with the catalyst at elevated temperature and pressure. The temperature is in the range of from 60 to 170° C., preferably 80 to 160° C., more preferably 100 to 150° C. The pressure may be in the range of from 1 to 200 bar (absolute), preferably of from 2 to 100 bar (absolute), more preferably of from 5 to 50 bar (absolute).

The organic phase comprising levulinic acid may further comprise one or more other ketones and/or aldehydes. Any ketone or aldehyde that can react with levulinic acid in an aldolcondensation reaction may be used. Examples of suitable aldehydes include formaldehyde, acetaldehyde, and butanal. Examples of suitable ketones include acetone, methyl ethyl ketone, and methyl isobutyl ketone.

If a ketone other than levulinic acid and/or an aldehyde is present in the organic phase, the organic phase preferably comprises at least 20 wt % levulinic acid, more preferably at least 50 wt %, even more preferably at least 80 wt %.

An organic phase of substantially pure levulinic acid is particularly suitable for the process according to the invention. Reference herein to substantially pure is to an organic phase comprising at least 90 wt % of levulinic acid, preferably at least 95 wt %.

The molar ratio between hydrogen supplied to the process and the total of levulinic acid, ketones and aldehydes in the organic phase is preferably in the range of from 0.2 to 10.0.

The liquid hourly space velocity of the organic phase comprising levulinic acid is typically in the range of from 0.01 to 10 liquid volumes of organic phase per hour per volume of catalyst, preferably of from 0.05 to 5, more preferably of from 0.1 to 3.

For the process according to the invention, any type of reactor that is suitable for a three-phase reaction may be used. Examples are a fixed catalyst bed trickle flow reactor or a slurry bubble column.

It has been found that in the process according to the invention, two molecules of levulinic acid are dimerised into the keto-di-carboxylic acid having the molecular structure of formula (1) by aldolcondensation followed by dehydration and hydrogenation. The IUPAC name of this di-carboxylic acid is 4-methyl-6-oxononanedioic acid. Since both carbon atoms adjacent to the carbonyl group in levulinic acid have a proton, the aldolcondensation can take place on two different carbon atoms and a second keto-di-carboxylic acid with the molecular structure of formula (2) is formed. The IUPAC name of this second keto-di-carboxylic acid is 3-acetyl-4-methylheptanedioic acid. Due to steric hindrance, less of the di-carboxylic acid according to molecular formula (2) than of that according to molecular formula (1) is formed.

It has been found that under the process conditions applied, part of the keto-di-carboxylic acids formed is hydrogenated to form di-carboxylic acids without a carbonyl group, i.e. 4-methylnonanedioic acid and 3-ethyl-4-methylheptanedioic acid.

Further, it has been found that in part of the aldol dimers formed from two molecules of levulinic acid, an internal ester bond is formed, resulting in a C₁₀ compound with a carbonyl group, a carboxyl group and a lactone group. Several of such compounds are formed, since two different aldol dimers are formed from levulinic acid and one of the aldol dimers can form two different 5-ring lactone groups. Thus, a compound with the molecular structure according to formula (3), i.e. 5-(2-methyl-5-oxotetrahydrofuran-2-yl)-4-oxopentanoic acid, is formed; a compound with the molecular structure according to formula (4), i.e. 3-(2-methyl-5-oxotetrahydrofuran-2-yl)-4-oxopentanoic acid; and a small amount of 3-(3-acetyl-2-methyl-5-oxotetrahydrofuran-2-yl)propanoic acid.

Under the process conditions applied, part of the C₁₀ compounds with a carbonyl group, a carboxyl group and a lactone group are hydrogenated to form the corresponding C₁₀ compound without the carbonyl group, i.e. 5-(2-methyl-5-oxotetrahydrofuran-2-yl)pentanoic acid, 3-(2-methyl-5-oxotetrahydrofuran-2-yl)pentanoic acid, and 3-(3-ethyl-2-methyl-5-oxotetrahydrofuran-2-yl)propanoic acid, respectively.

Under the reaction conditions of the process according to the invention, a small amount of the lactones according to formulas (3) and (4) may be further converted into a C₁₀ compound with two lactone groups. An internal ester bond is then formed between the enol form of the carbonyl group and the carboxyl group, followed by hydrogenation of the alkene group. The two resulting compounds with two lactone groups have the molecular structures of formulas (5) and (6), respectively.

It has been found that in the process according to the invention, a small part of the di-carboxylic acid according to formula (1) may be converted into a lactone by the formation of an internal ester bond between one of the carboxyl groups and the hydroxyl group of the enol form of the carbonyl group, followed by hydrogenation of the alkene group. The resulting compound, starting from the di-carboxylic acid according to formula (1) is 4-methyl-5-(5-oxotetrahydrofuran-2-yl)pentanoic acid (see molecular formula (7)). Starting from the di-carboxylic acid according to formula (2), a compound named 4-(2-methyl-5-oxotetrahydrofuran-3-yl)pentanoic acid is formed (see molecular formula (8)).

In case that not only levulinic acid, but also another ketone and/or aldehyde is present in the organic phase, not only levulinic acid dimers are formed, but also mono-carboxylic mixed dimers by aldolcondensation of one molecule of levulinic acid and one molecule of ketone or aldehyde followed by dehydration and hydrogenation, and dimers of two molecules of ketone or aldehyde.

Since the aldolcondensation can take place on the carbonyl carbon (only if the other ketone or aldehyde has an alpha proton) or on one of the two carbon atoms adjacent to the carbonyl group of levulinic acid, two or three different mixed dimers may be formed. In case of an asymmetric ketone with a proton on each of the two carbon atoms adjacent to the carbonyl group (i.e. a ketone with two different alpha protons), even four different mixed dimers may be formed. Some of the mixed dimers may be converted into a lactone by the formation of an internal ester bond between the enol form of the carbonyl group and the carboxyl group, followed by hydrogenation of the alkene group.

In the process according to the invention, the conversion of levulinic acid, ketones and aldehydes is typically in the range of from 10 to 50 wt %, usually between 20 and 40 wt %. This means that in the case of an organic phase of pure levulinic acid, a mixture mainly comprising levulinic acid and the di-carboxylic acid according to formula (1) is obtained. The mixture will typically also comprise smaller amounts of the further C₁₀ di-carboxylic acids described hereinabove and of the C₁₀ compounds with a carboxylic group and a lactone group described hereinabove. If a ketone other than levulinic acid and/or an aldehyde is present in the organic phase, the mixture obtained with the process further comprises the other ketone(s) and/or aldehyde(s), mixed dimers and, optionally, a dimer formed from two molecules of the ketone or aldehyde. If the other ketone is acetone, the dimer formed from two acetone molecules is methyl isobutyl ketone (MIBK). MIBK may be recycled to the process to form part of the organic phase.

The levulinic acid dimers and the mixed dimers obtainable in the process according to the invention may be used as such, for example as solvent for biomass liquefaction. Alternatively, the dimers can be used as intermediates for other useful compounds. The di-carboxylic acids formed from two molecules of levulinic acid may for example suitably be applied as building block for polymers, in particular as co-monomer in polyesters or polyamides. The di-carboxylic acids and the mono-carboxylic dimers may also be converted into the corresponding di- or mono-esters by reacting them with an alcohol under esterifying conditions.

Di-esters of the di-carboxylic acids according to molecular formulas (1) and (2) have the general molecular formulas (9) and (10), respectively. R₁ and R₂ are, independently, an organic radical which is covalently connected to the oxygen atom by a carbon atom. The organic radical may for example be a substituted or un-substituted alkyl, alkene, aryl, alkoxy, or heterocyclic group. R₁ and R₂ may be a straight, branched or cyclic group. R₁ and R₂ may be part of a single structure, thus forming a cyclic ester. R₁ and R₂ are preferably a straight or branched alkyl group having up to 22 carbon atoms, more preferably up to 10 carbon atoms, even more preferably a methyl, ethyl, butyl or isobutyl group. A di-ester wherein both R₁ and R₂ are ethyl is particularly preferred.

Di-esters wherein R₁ and R₂ are not equal to each other may for example be obtained by first forming a mono-ester by esterifying the di-carboxylic acids with a limited amount of a first alcohol and then forming a di-ester by continuing the esterification with a second alcohol. Alternatively, such a di-ester may be formed by transesterification of a di-ester with two equal alcohol fragments.

The di-esters may for example be used as hydraulic fluid, in heating oil, as co-monomer is polymers such as polyesters or polyamides, as solvent, in particular as solvent for biomass liquefaction, and as fuel component in for example diesel fuel. An advantage of the use of the di-esters in diesel fuel is that they have lubricating properties. A further advantage is that the di-ester may be derived from biomass.

Esters of the mono-carboxylic acid dimers obtainable in the process according to the invention, in particular esters of the C₁₀ compounds with a lactone group, may be used as solvent for biomass liquefaction. Also the mono-carboxylic acid dimers as such and the dimers with two lactone groups according to formula (5) and (6) may be used as solvent for biomass liquefaction.

If, in the process according to the invention, the organic phase comprises no aldehyde and no ketone other than levulinic acid, the whole effluent of the process, i.e. a mixture mainly comprising levulinic acid and 4-methyl-6-oxononanedioic acid (the di-carboxylic acid according to formula (1)), may be reacted with an alcohol under esterifying conditions to yield a mixture comprising an ester of levulinic acid and a di-ester of 4-methyl-6-oxononanedioic acid. Such ester mixture typically also comprises smaller amounts of the esters of the other C₁₀ di-carboxylic acids and carboxyl-lactones that have been described hereinabove.

The alcohol used for esterification of the dimers or of the mixture comprising levulinic acid and dimers is preferably an alkyl alcohol comprising 1 to 10 carbon atoms, more preferably methanol, ethanol, n-butanol, or 2-methyl-1-propanol.

If the ester mixture or the di-esters or esters according to the invention are used in fuel, they are preferably used in a concentration up to 5 wt % based on the weight of the total fuel.

### EXAMPLE

The process according to the invention will be further illustrated by means of the following non-limiting example.

A reactor was filled with 26.6 g of beads of an industrial grade palladium-doped strongly acidic macroreticular ion-exchange resin catalyst (AMBERLYST® CH 28, ex. Rohm and Haas Company). The catalyst comprises 0.7 wt % Pd on a macroreticular styrene divinylbenzene co-polymer with sulphonic acid groups. The empty space above the catalyst bed was filled with 0.8 mm diameter silicon carbide particles. Catalyst and silicon carbide particles were fixed between plugs of ceramic wool.

The reactor was pressurised with hydrogen to a pressure of 20 bar g and brought to a temperature of 130° C. An organic phase comprising 98 wt % levulinic acid was pre-heated to 35° C. in order to melt the levulinic acid and then fed to the reactor at a weight hourly velocity of 0.5 g/g catalyst/h. Hydrogen was fed to the reactor at a hydrogen/organic phase ratio of 1.0 L hydrogen per gram organic phase (hydrogen/levulinic acid molar ratio is 5.2).

The effluent of the process was analysed both by GC-MS (after silylation of the effluent) and NMR spectroscopy (after esterification of the effluent with ethanol). These analyses showed that the effluent contained 70 wt % levulinic acid, 26 wt % of levulinic acid dimers, 2 wt % of C₅ compounds (mainly pentanoic acid and gamma valerolactone) and 2 wt % unknown compounds. Table 1 shows the amounts of the individual levulinic acid dimers identified in the reaction mixture.

TABLE 1
Levulinic acid dimers identified in the reaction mixture.

| | Yield | Molecular |
|---|---|---|
| Levulinic acid dimer | (wt %) | formula |
| 4-methyl-6-oxononanedioic acid | 14.3 | 1 |
| 3-acetyl-4-methylheptanedioic acid | 5.9 | 2 |
| 4-methylnonanedioic acid | 0.4 | |
| 3-ethyl-4-methylheptanedioic acid | 0.8 | |
| 5-(2-methyl-5-oxotetrahydrofuran-2-yl)-4- | 0.3 | 3 |
| oxopentanoic acid | | |
| 3-(2-methyl-5-oxotetrahydrofuran-2-yl)-4- | 0.4 | 4 |
| oxopentanoic acid | | |
| 3-(3-acetyl-2-methyl-5-oxotetra-hydrofuran-2- | <0.05 | |
| yl)propanoic acid | | |
| 5-(2-methyl-5-oxotetrahydrofuran-2-yl)pentanoic | 0.6 | |
| acid | | |
| 3-(2-methyl-5-oxotetrahydrofuran-2-yl)pentanoic | 0.8 | |
| acid | | |
| 3-(3-ethyl-2-methyl-5-oxotetra-hydrofuran-2- | 2.5 | |
| yl)propanoic acid | | |

What is claimed is:

1. A process for dimerisation of levulinic acid, wherein an organic phase comprising levulinic acid is contacted, in the presence of hydrogen, with a strong acidic heterogeneous catalyst comprising a hydrogenating metal, at a temperature in the range of from 60 to 170° C. and a pressure in the range of from 1 to 200 bar (absolute).

2. A process according to claim 1, wherein the hydrogenating metal is a Group VIII metal.

3. A process according to claim 1, wherein the catalyst further comprises a strong acidic ion-exchange resin.

4. A process according to claim 1, wherein the organic phase is contacted with the catalyst at a temperature in the range of from 80 to 160° C.

5. A process according to claim 1, wherein the pressure is in the range of from 2 to 100 bar (absolute).

6. A process according to claim 1, wherein the organic phase comprises levulinic acid and one or more ketones or aldehydes.

7. A process according to claim 6, wherein the organic phase comprises levulinic acid and one or more aldehydes selected from the group consisting of formaldehyde, acetaldehyde, and butanal.

8. A process according to claim 6, wherein the organic phase comprises levulinic acid and one or more ketones selected from the group consisting of acetone, methyl ethyl ketone, and methyl isobutyl ketone.

9. A process according to claim 6, wherein the organic phase comprises at least 20 wt % of levulinic acid.

10. A process according to claim 1, wherein the organic phase is substantially pure levulinic acid.

11. A di-carboxylic acid having the molecular structure according to formula

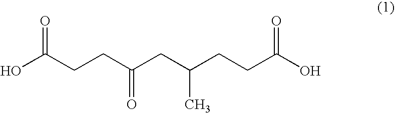

12. A di-carboxylic acid having the molecular structure according to formula

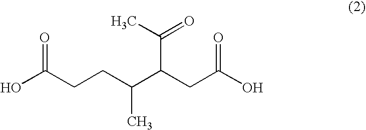

13. A C₁₀ compound with a carboxylic group and a lactone group having the molecular structure according to formula

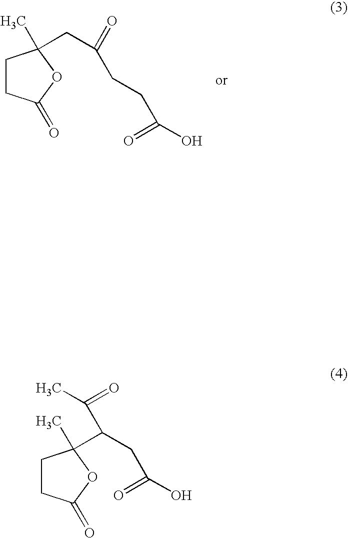

14. A C₁₀ compound with two lactone groups having the molecular structure according to formula

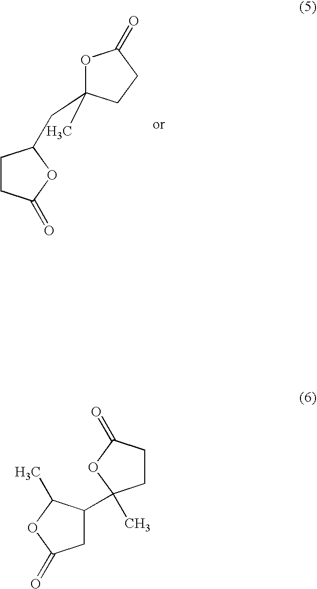

15. A C₁₀ compound with a carboxylic group and a lactone group having the molecular structure according to formula

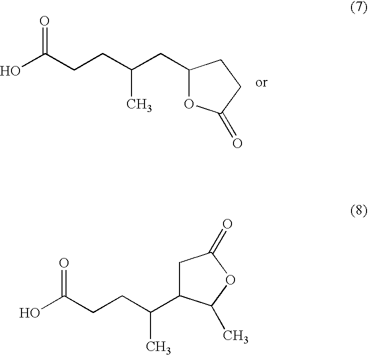

16. A mixed dimer obtainable by a process according to claim 6, wherein the mixed dimer is formed from one molecule of levulinic acid and one molecule of a ketone selected from the group consisting of acetone, methyl ethyl ketone, and methyl isobutyl ketone, or the mixed dimer is formed from one molecule of levulinic acid and one molecule of an aldehyde selected from the group consisting of formaldehyde, acetaldehyde, and butanal.

17. A mixture comprising levulinic acid and the di-carboxylic acid according to claim 11.

18. A di-ester having the general molecular structure according to formula 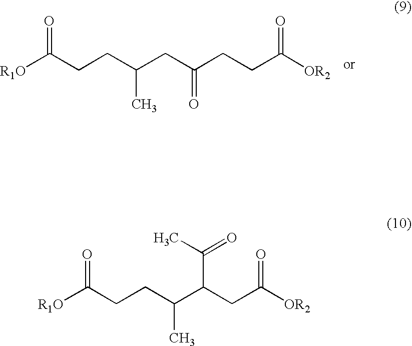
wherein R₁ and R₂ are, independently, an organic radical which is covalently connected to the oxygen atom by a carbon atom.

19. A di-ester according to claim 18, wherein R₁ and R₂ are, independently, a straight or branched alkyl group having up to 22 carbon atoms.

20. An ester obtainable by reacting a C₁₀ compound comprising a carboxyl group and a lactone group, or by reacting a mixed dimer, with an alcohol under esterifying conditions.

21. A mixture comprising an ester of levulinic acid and a di-ester of the di-carboxylic acid according to claim 11, obtainable by reacting a mixture comprising the levulinic acid and the di-carboxylic acid with an alcohol under esterifying conditions wherein the alcohol comprises an alkyl alcohol comprising 1 to 10 carbon atoms.
